(19) United States
(12) Patent Application Publication
(10) Pub. No.: US 2020/0042845 A1
(43) Pub. Date: Feb. 6, 2020

(54) SYSTEM FOR AND METHODS OF SECURE ONLINE PERSONAL DATA STORAGE AND ACCESS

(71) Applicant: Hans Christian Rasmussen, Trent Woods, NC (US)

(72) Inventor: Hans Christian Rasmussen, Trent Woods, NC (US)

(21) Appl. No.: 16/055,948

(22) Filed: Aug. 6, 2018

(51) Int. Cl. G06K 19/06; G16H 10/65; G06F 21/62; G06F 17/30

(52) U.S. Cl. CPC G06K 19/06037; G06F 17/30991; G06F 21/6245; G16H 10/65

(57) ABSTRACT

A system for and methods of secure online personal data storage and access, wherein member users may enter any type of information and wherein some portion of the entered information can be marked as public. Further, each member user may have a membership card with an encoded identifier, such as a quick response (QR) code, barcode, RFID tag, or alphanumeric code thereon and wherein a third party can scan the encoded identifier and view the public information of the member user.

### TECHNICAL FIELD

The presently disclosed subject matter relates generally to alerting or otherwise informing one person about another person's medical or other information and more particularly to a system for and methods of secure online personal data storage and access.

### BACKGROUND

First responders (e.g., emergency medical services (EMS), police, firefighters) face many challenges during medical emergencies. For example, one challenge for EMS is gaining access to accurate and up-to-date personal health and emergency contact information. Medical identification (ID) devices exist for alerting or otherwise informing one person (e.g., a first responder) about another person's medical information. Medical IDs can be used to record and communicate critical medical information in the event that the wearer is unable to do so. For example, medical ID bracelets, medical ID key chains, medical ID cards, medical ID jewelry (e.g., necklace), medical ID lanyards, and the like are currently in use for assisting first responders. Such devices can be a fast and effective way for pre-hospital emergency personnel or any healthcare practitioner to gain secure access to potentially lifesaving information.

However, a drawback of current systems that support medical ID devices is that it may be difficult for users to manage the content of their medical information and to manage the information that is accessible by third parties (e.g., first responders). Another drawback of current systems that support medical ID devices is that the type of information that can be stored, processed, and/or conveyed is limited.

Additionally, police officers are often unaware of mental health and substance abuse issues that require specialized contact approaches. Many officers have specialized training to deal with these populations, but few reliable systems exist to identify these citizens quickly in the event of an emergency.

### SUMMARY

In one embodiment, the invention provides a system for secure online personal data storage and access. The system may include a hardware processor; and a memory device, the memory device storing instructions, the instructions when executed causing the hardware processor to perform operations, the operations may include receiving a member unique identification code, wherein the member unique identification code is accessed from an encoded identifier device; querying an electronic database for member data associated with the member unique identification code; retrieving at least a portion of the member data associated with the member unique identification code; and displaying the retrieved member data. The system may further include an encoded identifier on or in the encoded identifier device, wherein the encoded identifier is associated with the member unique identification code. The system may further include a third-party user device configured to read the encoded identifier to access the member unique identification code. The encoded identifier may include at least one of a quick response (QR) code, barcode, RFID tag, or alphanumeric code. The third-party user device is preferably configured to receive and display the retrieved member data. The encoded identifier device may include a member user card, wherein the encoded identifier may include at least one of a quick response (QR) code, barcode, RFID tag, or alphanumeric code. The system may further include a member user device, wherein the member user device is preferably configured to one or more of input, edit, and/or access the member data. The operations may further include sending the retrieved member data to the third-party user device. The operations may further include designating certain portions of the member data as public, and wherein only the member data designated as public is displayable on the third party device.

In another embodiment, a system for secure online personal data storage and access may include an encoded identifier device; a third-party device configured to read an encoded identifier on or in the encoded identifier device; a hardware processor; and a memory device, the memory device storing instructions, the instructions when executed causing the hardware processor to perform operations, the operations may include receiving a member unique identification code from the third-party device, wherein the member unique identification code is accessed by the third-party device by reading the encoded identifier of the encoded identifier device; querying an electronic database for member data associated with the member unique identification code; retrieving at least a portion of the member data associated with the member identification code; and displaying the retrieved member data to the third-party device. The encoded identifier device may include an encoded identifier comprising at least one of a quick response (QR) code, barcode, RFID tag, or alphanumeric code. The encoded identifier device may include a member user card, wherein the encoded identifier may include at least one of a quick response (QR) code, barcode, RFID tag, or alphanumeric code. The system may further include a member user device, wherein the member user device is preferably configured to one or more of input, edit, and/or access the member data. The operations may further include designating certain portions of the member data as public, and wherein only the member data designated as public is displayed to the third-party device.

In yet another embodiment, a method of secure online personal data storage and access is provided. The method may include receiving, by a server, a service request sent via the Internet from a client device, the service request requesting a cloud-based data retrieval service performed on behalf of the client device, the service request specifying a member unique identification code; querying, by the server, an electronic database for member data associated with the member unique identification code, the electronic database electronically associating the member unique identification code with the associated member data; retrieving, by the server, at least a portion of the associated member data; sending, by the server, the retrieved member data via the Internet to the client device; and displaying the retrieved member data. The member unique identification code is preferably accessed from an encoded identifier disposed on or in an encoded identifier device. The client device may include a third-party device configured to read the encoded identifier of the encoded identifier device. The retrieved member data may include only member data designated as public. The encoded identifier may include at least one of a quick response (QR) code, barcode, RFID tag, or alphanumeric code. The client device may include a member user device, wherein the member user device is configured to one or more of input, edit, and/or access the member data.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 illustrates a block diagram of an example of the presently disclosed information access system for secure online personal data storage and access;

FIG. 2 illustrates a block diagram of an example of the cardholder data stored in the presently disclosed information access system;

FIG. 3 illustrates a plan view of an example a membership card of the presently disclosed information access system;

FIG. 4 illustrates an example of a third-party access menu, which is one example of the graphical user interface of the presently disclosed information access system.

FIG. 5A and FIG. 5B show an example of the viewable cardholder information that can be displayed to third parties of the presently disclosed information access system;

FIG. 6 through FIG. 21 show examples of various other screens and/or menus of the graphical user interface of the presently disclosed information access system;

FIG. 22 illustrates a flow diagram of an example of a method of a cardholder user using the presently disclosed information access system; and

FIG. 23 illustrates a flow diagram of an example of a method of a third-party user using the presently disclosed information access system.

### DETAILED DESCRIPTION

The presently disclosed subject matter now will be described more fully hereinafter with reference to the accompanying Drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides an information access system for and methods of secure online personal data storage and access. Namely, an information access system and information access application are provided, wherein users may enter any type of information and wherein some portion of the information can be designated public information and another portion of the information can be designated private information. Generally, in the information access system, all information is classified private by default unless specifically marked public by the user. Further, each user has a membership card with an encoded identifier, such as a quick response (QR) code or barcode, printed thereon and wherein a third party can scan the encoded identifier and view the public information of the user.

In some embodiments, the presently disclosed information access system for and methods of secure online personal data storage and access can be used in a health application and more particularly in a first responder application. In this embodiment, in a medical emergency, a first responder may scan the encoded identifier on the membership card of the user seeking assistance. In so doing, the public profile of the user seeking assistance is displayed to the first responder and wherein the public profile includes health information that may be useful for treatment, such as, but not limited to, medical conditions, physicians, medications, medical history, and the like.

In some embodiments of the presently disclosed information access system for and methods of secure online personal data storage and access, while generally all information is classified private by default, the user may selectively mark certain portions of his/her information as public information that can be viewed by any third party.

In some embodiments, whereas the presently disclosed information access system for and methods of secure online personal data storage and access may be tailored for use in, for example, a health application and more particularly for use in a first responder application, any other types of information (albeit unrelated to health) may be stored, processed, and/or conveyed using the information access system.

In some embodiments, the information access system includes a web-based information access application hosted on an application server.

Further, a method is provided of a cardholder user using the information access system and information access application. Additionally, a method is provided of a third-party user (e.g., a first responder) using the information access system and information access application.

While the presently disclosed information access system for secure online personal data storage and access is described hereinbelow with reference of a health application and more particularly to a first responder application, the presently disclosed information access system is not limited to such applications. The use of the presently disclosed information access system in a health application and more particularly to a first responder application is exemplary only. The presently disclosed information access system can be tailored for any other applications, such as but not limited to, medical offices, schools, daycare centers, assisted living facilities, any other types of businesses as a means of maintaining clients' records, and the like.

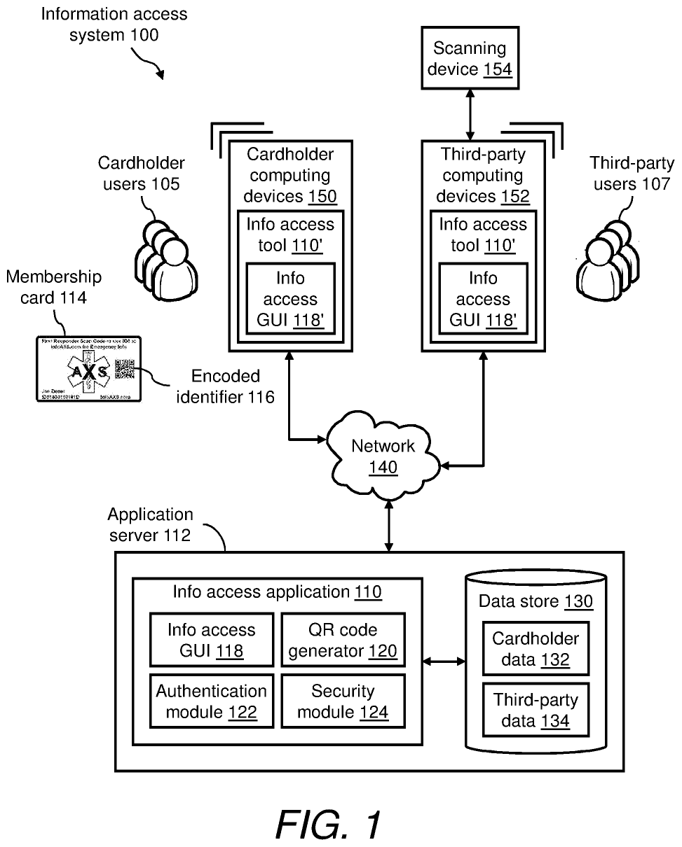

Referring now to FIG. 1 is a block diagram of an example of the presently disclosed information access system 100 for secure online personal data storage and access. Information access system 100 includes an information access application 110 running on an application server 112.

Application server 112 can be any networked computing configuration. For example, information access system 100, and more particularly info access application 110 on application server 112, may support a cloud computing environment. In a cloud computing environment, application server 112 is the cloud server. Further, info access application 110 is not limited to running on one application server 112 only. Information access system 100 may include multiple application servers 112 (or cloud servers) in order to ensure high-availability of computing resources.

Information access system 100 and info access application 110 support both cardholder users 105 and third-party users 107. Cardholder users 105 can be any individuals. Third-party users 107 can be any users and/or entities that are authorized to view certain information about cardholder users 105. In medical applications, third-party users 107 can be, for example, first responders (e.g., EMS, police, firefighters) and/or any healthcare practitioners, such as doctors, nurses, any health caregivers, pharmacists, family members of cardholder users 105, and the like. In other applications, third-party users 107 can be, for example, personnel of schools, daycare centers, assisted living facilities, business, and any other entities wherein information access system 100 can be used as a means of maintaining client records.

Generally, info access application 110 is a web-based application that provides a mechanism for storing and managing personal information (e.g., health and/or any other information) of cardholder users 105 and providing a medical ID device, such as, but not limited to, a membership card 114 that has the user's unique ID number and/or an encoded identifier 116 printed thereon. Encoded identifier 116 may be any mark that includes a machine-readable code. In one example, the information encoded into encoded identifier 116 is the web address (i.e., the URL) of information access system 100 and the ID number of a certain cardholder user 105. Encoded identifier 116 may be, for example, a scannable quick response (QR) code, a scannable barcode, a readable magnetic strip, a readable radio-frequency identification (RFID) chip, and the like. In some embodiments, membership card 114 shows the user's unique ID number only and is absent encoded identifier 116. In one example, in a medical emergency situation, the encoded identifier 116 on membership card 114 can be scanned by a third-party user 107, wherein, using the information encoded in the encoded identifier 116, health and/or any other information of the cardholder user 105 can be accessed and viewed by the third-party user 107.

Info access application 110 at application server 112 manages the overall operations of information access system 100. Info access application 110 includes, for example, but is not limited to, an info access graphical user interface (GUI) 118, a QR code generator 120, an authentication module 122, and a security module 124.

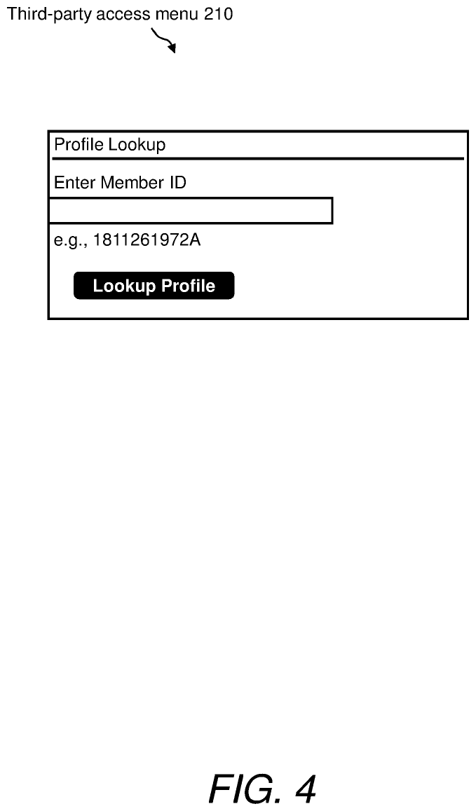

Info access GUI 118 can be any collection and/or design of screens and/or menus that can be displayed on a computing device for accessing info access application 110. More details of examples of screens and/or menus of info access GUI 118 are shown and described hereinbelow with reference to FIG. 4 through FIG. 21.

QR code generator 120 can be a QR code generator capable of generating an encoded identifier 116 for each of the cardholder users 105. Again, the information encoded into encoded identifier 116 can be the web address (e.g., www.infoaxs.com) of information access system 100 and the unique ID number of any certain cardholder user 105. In one example, the ID number may be an alphanumeric number, such as 1803110181D or 1806210173A. In one example, QR code generator 120 may also perform the function of generating the alphanumeric ID numbers.

Authentication module 122 is used to manage the authentication process of cardholder users 105. For example, authentication module 122 may compare a submitted username and password with a list of registered usernames and passwords of cardholder users 105 and/or third-party users 107. The list of usernames and passwords may be stored in and accessed from a database (e.g., a data store 130). If authentication module 122 is able to match the submitted username and password with a registered username and password, then the user may be granted a level of access to info access application 110. Otherwise, the user is not granted access to info access application 110.

Security module 124 may perform various application security functions within information access system 100. For example, security module 124 can be used to implement network policies and controls, work with authentication module 122 with respect to access control (e.g., to authorize users while at the same time blocking malicious actors), provide antivirus and antimalware protection, provide data loss protection, manage firewalls, provide intrusion protection, and the like. Security module 124 may use various security techniques, such as encryption, secure hashtags (or hash tags), and the like. Further, security module 124 may be used to perform any system security functions with respect to keeping the contents of the data store 130 secure, for example, to ensure compliance with patient health data privacy laws, such as the Health Insurance Portability and Accountability Act (HIPAA).

Data store 130 at application server 112 can be, for example, data repositories (like databases) and/or flat files that can store data. Further, information access system 100 is not limited to one data store 130 only. Information access system 100 may include multiple data stores 130. Data store 130 at application server 112 is used, for example, to store any information about cardholder users 105 as well as any other application data. For example, cardholder data 132 and third-party data 134 is stored at data store 130. Namely, cardholder data 132 includes a record of information for each of the cardholder users 105. Third-party data 134 includes a record of information for certain third-party users 107.

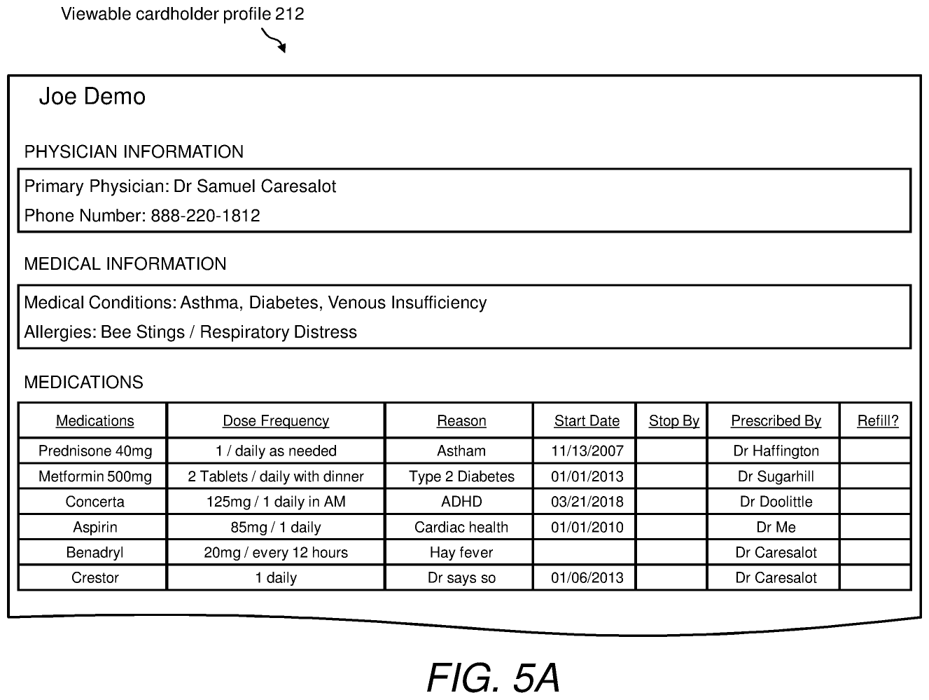

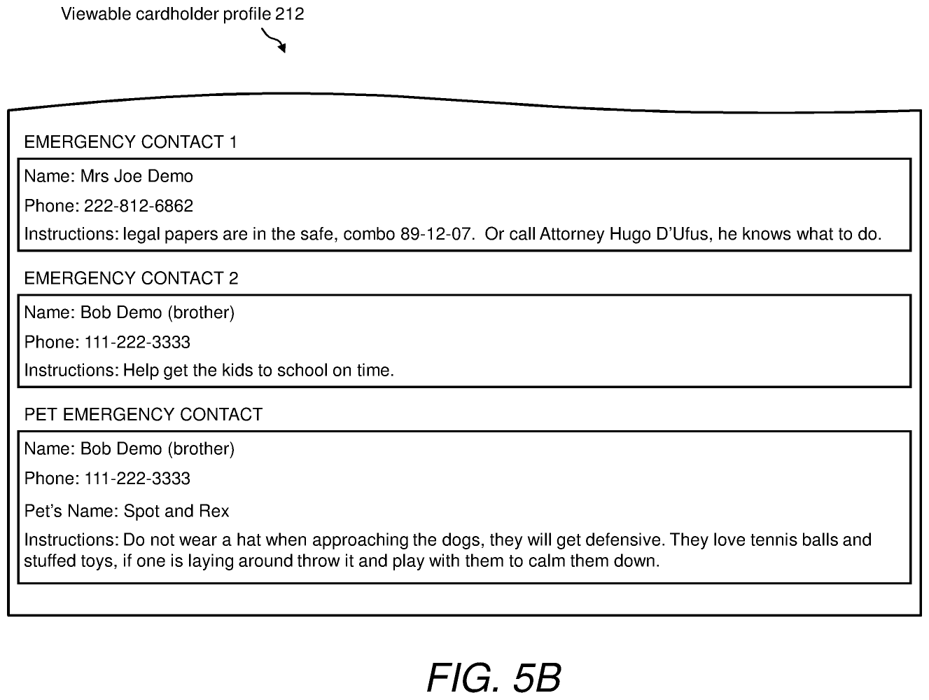

For each cardholder user 105, cardholder data 132 may include, for example, user profile information (e.g., account information, personal information, contact information), payment information (e.g., credit card and/or bank information), health information (e.g. medical conditions, physicians, medications, medical history), family member information, pets information, and emergency contact information.

Whereas generally third-party users 107 can access public information (see FIG. 5A and FIG. 5B) about cardholder users 105 without registering to, signing up to, or being a member of information access system 100, there may be circumstances that require third-party user signup. For example, a certain third-party user 107 may be granted access to a certain cardholder user 105's information to, for example, insert clinical information on a designated page through a separate portal/password. In this example, the third-party user 107 will not have full access unless the cardholder user 105 makes their master password available. Accordingly, third-party data 134 may include, for example, user profile information (e.g., account information, personal information, contact information) of certain third-party users 107.

Further, a feature of info access application 110 is that it is not limited to health applications and health information. Cardholder users 105 can use info access application 110 to store, process, and/or convey any type of information, such as, but not limited to, journal entries, other accounts or passwords information, any textual information and/or digital information. Additionally, cardholder users 105 can use info access application 110 to store, process, and/or convey file uploads that may include medical records, hospital discharge summaries, and the like. Further, while generally in info access application 110, all information is classified private by default, another feature of info access application 110 is that cardholder users 105 can mark certain information as public wherein public information may be accessible and viewable by third-party users 107.

Info access application 110 at application server 112 is accessible via a network 140. Network 140 may be, for example, a local area network (LAN), a wide area network (WAN), and/or a cellular network for connecting to the Internet or to an Intranet.

Information access system 100 may further include cardholder computing device(s) 150 that are used by cardholder users 105 to access info access application 110. Similarly, information access system 100 may further include third-party computing device(s) 152 that are used by third-party users 107 to access info access application 110. Cardholder computing devices 150 and third-party computing devices 152 are preferably computing devices capable of processing program instructions and capable of connecting to a network. For example, cardholder computing devices 150 and third-party computing devices 152 may be desktop computers, laptop computers, tablet devices, mobile phones or smartphones, smart watches, and the like.

In one example, information access system 100 is using a client-server type of architecture. Namely, an info access tool 110′ is installed and running on each of the cardholder computing devices 150 and third-party computing devices 152, wherein info access tool 110′ is the counterpart to info access application 110 at application server 112. Info access tool 110′ can be a browser-based tool, a desktop application, and/or a mobile app depending on the type of computing device on which it is running. Accordingly, info access tool 110′ includes info access GUI 118′ by which a user interacts with info access application 110 at application server 112. In particular, on third-party computing devices 152, info access tool 110′ can be a web browser for accessing the website of information access system 100.

Further, each of the third-party computing devices 152 preferably has a scanning device 154. Scanning device 154 is used by a third-party user 107 to scan the encoded identifier 116 on membership card 114 and extract the encoded information thereof. In one example, scanning device 154 can be any optical scanning device capable of scanning QR codes and/or barcodes. In another example, scanning device 154 can be credit card-type of magnetic strip reader. In yet another example, scanning device 154 can be an RFID reader.

In the case that the third-party computing device 152 is a desktop or laptop computer, scanning device 154 can be a device that is separate from the third-party computing device 152. In the case that the third-party computing device 152 may be a smartphone, tablet device, or smartwatch, scanning device 154 can be a separate device or a built-in optical scanning device capable of scanning QR codes and/or barcodes. For example, the Apple iPhone and Android phone may use their camera and a built-in QR code reader or barcode reader app. In another example, a QR code scanner is built in to the Chrome app, or the like.

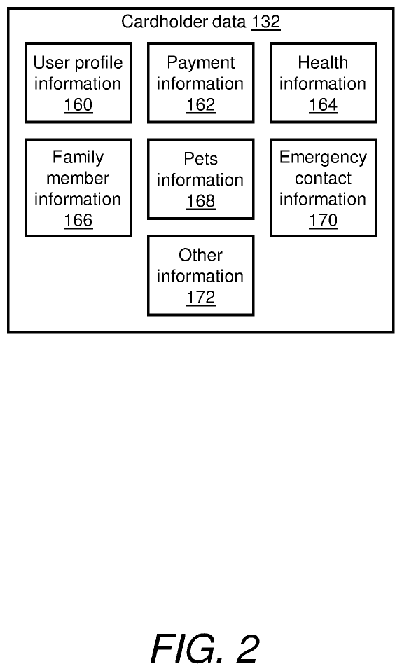

Referring now to FIG. 2 is a block diagram of an example of cardholder data 132 stored in the presently disclosed information access system 100. Cardholder data 132 may include, for example, user profile information 160 (e.g., account information, personal information, contact information), payment information 162 (e.g., credit card and/or bank information, coupons), health information 164 (e.g., medical conditions, physicians, medications, medical history), family member information 166, pets information 168, and emergency contact information 170 (e.g., emergency contacts for self, children, pets).

Info access application 110 can be used to store, process, and/or convey any type of information. Accordingly, cardholder data 132 may include other information 172. Other information 172 may include, but is not limited to, journal entries, records of other accounts and/or passwords, any textual and/or digital information, and the like.

Further, cardholder data 132 includes both “public” information and “private” information. In information access system 100, public information means any information in cardholder data 132 that cardholder users 105 have marked as viewable by third-party users 107. By contrast, private information means any information in cardholder data 132 that cardholder users 105 have marked as not viewable by third-party users 107 (i.e., information this is blocked from view), or, for example, any information not specifically marked by cardholder user 105 as public information. That is, private information is only viewable by cardholder users 105. Generally, in information access system 100, all information is classified private by default unless specifically marked public by the user.

Again, the presently disclosed information access system 100 is not limited to medical applications and more particularly to a first responder application. Accordingly, the information stored in cardholder data 132 can vary depending on the type of application in which the information access system 100 is used. For example, the information stored in cardholder data 132 can be tailored for any other applications, such as but not limited to, medical offices, schools, daycare centers, assisted living facilities, any other types of businesses as a means of maintaining clients' records, and the like

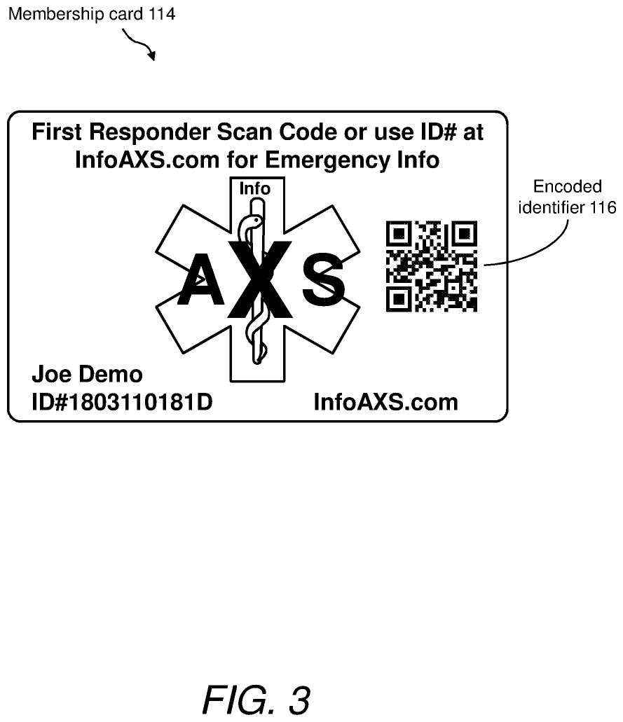

Referring now to FIG. 3 is a plan view of an example of membership card 114 of the presently disclosed information access system 100. Membership card 114 can be a plastic card that is preferably standard credit card size. For example, membership card 114 may be about 3⅜ inches×2⅛ inches (standard size CR80). Printed on membership card 114 may be, for example, the name and ID number of the cardholder user 105, along with encoded identifier 116 (e.g., QR code). Again, encoded identifier 116 may be encoded with the web address (e.g., www.infoaxs.com) of information access system 100 and the unique ID number (e.g., 1803110181D) of the cardholder user 105.

Information access system 100 is not limited to using membership cards 114. In other embodiments, encoded identifier 116 can be provided on other objects, such as, but not limited to, jewelry (e.g., necklace, bracelets), key chains, lanyards, stickers that can be affixed to other objects, RFID devices, and the like.

For example, FIG. 4 shows a third-party access menu 210, which is one example of info access GUI 118 of information access system 100. In operation, when a third-party user 107 scans the encoded identifier 116 on the membership card 114 using his/her scanning device 154, info access tool 110′ processes the information in encoded identifier 116. Then, the info access GUI 118′ automatically redirects to third-party access menu 210 and the unique ID number of the cardholder user 105 autofills (or auto-fills) the “Enter Member ID” field. Optionally, third-party user 107 manually enters the ID number into the “Enter Member ID” field. Then, third-party user 107 presses the “Lookup Profile” pushbutton to view the information of the cardholder user 105 of interest. For example, upon pressing the “Lookup Profile” pushbutton, a viewable cardholder profile 212 is displayed in info access GUI 118′. An example of viewable cardholder profile 212 is shown in FIG. 5A and FIG. 5B.

Referring now to FIG. 5A and FIG. 5B, a viewable cardholder profile 212 for Joe Demo is shown. The contents of viewable cardholder profile 212 is compiled from “public” information only of Joe Demo, wherein “private” information is not compiled and presented for viewing. In this example, Joe Demo's physician information, medical information, medications information, emergency contact information, and pet contact information is displayed to third-party users 107 and is also printable. This is because, in info access application 110, Joe Demo has marked his physician information, medical information, medications information, emergency contact information, and pet contact information as “public” information. Other information, such as records of accounts/passwords, journal entries, and the like, is classified private by default and is therefore not included in viewable cardholder profile 212.

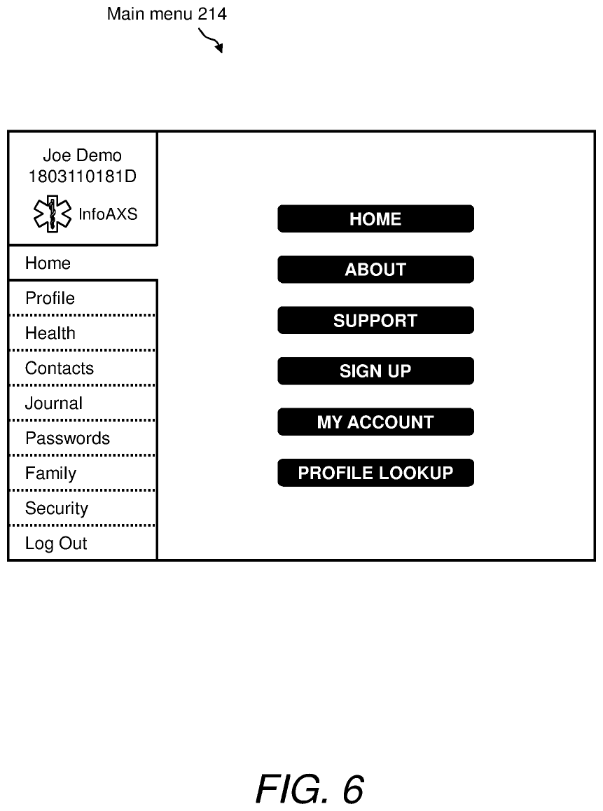

Referring now to FIG. 6 through FIG. 21 is examples of various other screens and/or menus of info access GUI 118 of the presently disclosed information access system 100. For example, FIG. 6 shows a main menu 214 of info access GUI 118. In this example, main menu 214 includes a home tab, a profile tab, a health tab, a contacts tab, a journal tab, a passwords tab, a family tab, a security tab, and a log out tab. However, these tabs are exemplary only. Main menu 214 of info access GUI 118 can include any number of tabs and any tab names.

In the view shown in FIG. 6, the home tab in main menu 214 is selected and the viewing panel shows example pushbuttons: HOME, ABOUT, SUPPORT, SIGN UP, MY ACCOUNT, and PROFILE LOOKUP. In one example, selecting the PROFILE LOOKUP pushbutton brings the user to third-party access menu 210 shown in FIG. 4.

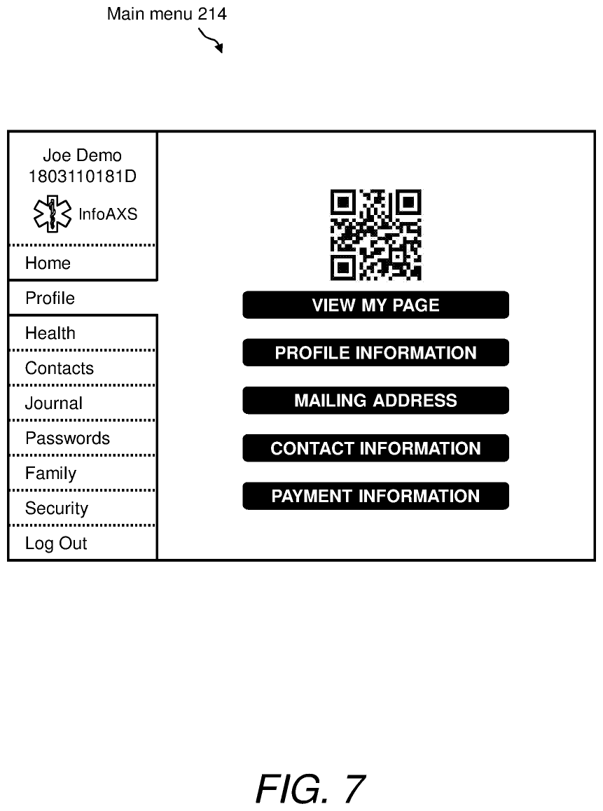

In the view shown in FIG. 7, the profile tab of main menu 214 is selected and the viewing panel shows example pushbuttons: VIEW MY PAGE, PROFILE INFORMATION, MAILING ADDRESS, CONTACT INFORMATION, and PAYMENT INFORMATION.

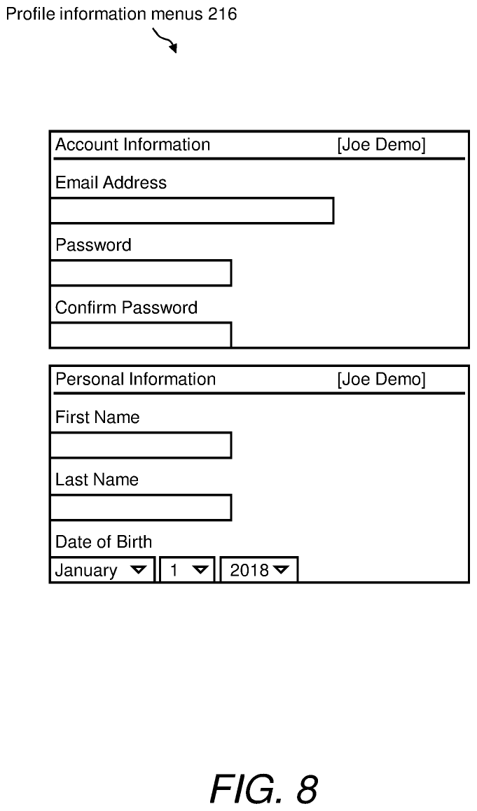

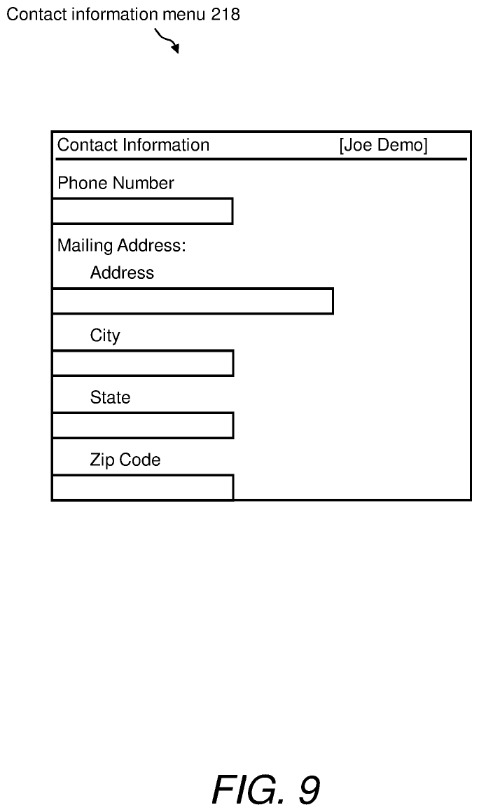

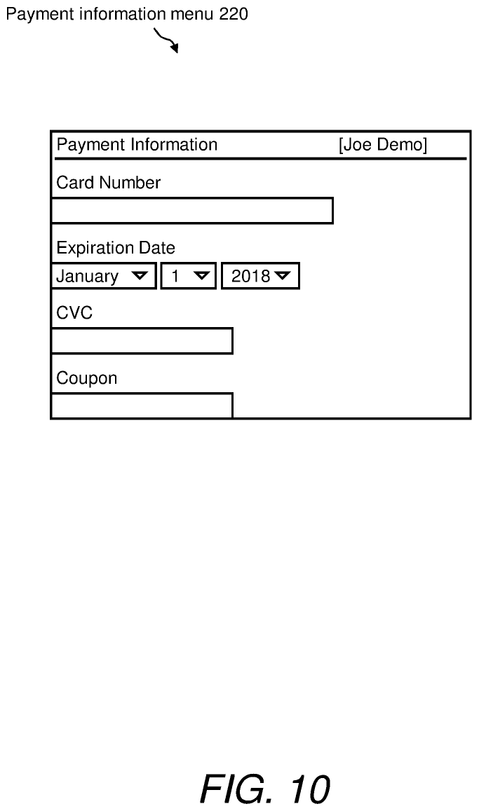

The VIEW MY PAGE pushbutton allows the user to see an example of what is displayed to third-party users 107, such as viewable cardholder profile 212 shown in FIG. 5A and FIG. 5B. Pressing the PROFILE INFORMATION pushbutton launches, for example, profile information menus 216 shown in FIG. 8, contact information menu 218 shown in FIG. 9, and payment information menu 220 shown in FIG. 10. Profile information menus 216 allow users to enter, for example, account information and personal information. Payment information menu 220 allows users to enter, for example, account information and personal information. Contact information menu 218 allows users to enter, for example, credit card information and/or coupon information. In one example, when a cardholder user 105 creates an account in information access system 100, the user is cycled through profile information menus 216, contact information menu 218, and payment information menu 220.

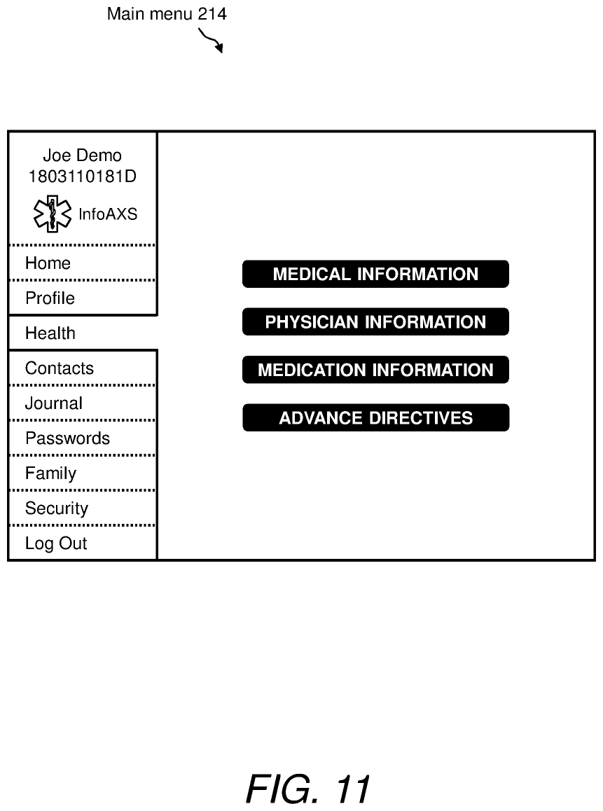

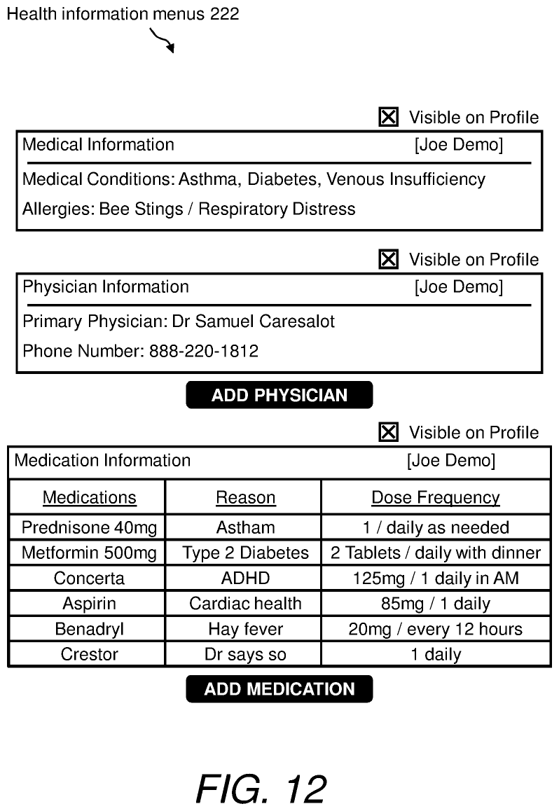

In the view shown in FIG. 11, the health tab in main menu 214 is selected and the viewing panel shows example pushbuttons: MEDICAL INFORMATION, PHYSICIAN INFORMATION, MEDICATION INFORMATION, and ADVANCED DIRECTIVES. Referring now to FIG. 12, a set of health information menus 222 are shown. For example, a medical information panel that correlates to the MEDICAL INFORMATION pushbutton, a medical information panel that correlates to the PHYSICIAN INFORMATION pushbutton, and a medical information panel that correlates to the MEDICATION INFORMATION pushbutton. Using health information menus 222 a user may enter his/her health information. Further, each panel preferably has a “Visible on Profile” check box. The “Visible on Profile” check box can be used to mark the information as “public.” That is, when the “Visible on Profile” check box is checked, then the information in the panel is public and viewable. By contrast, when the “Visible on Profile” check box is not checked, then the information in the panel is private and not viewable.

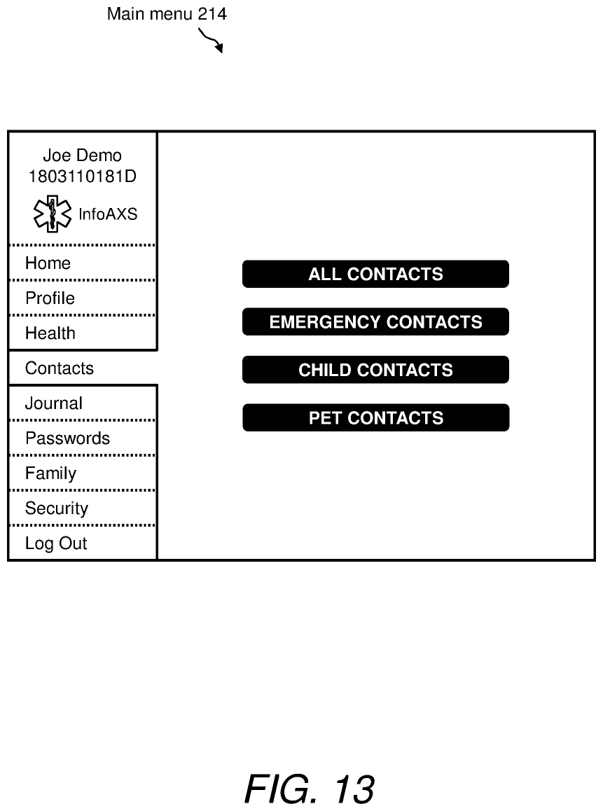

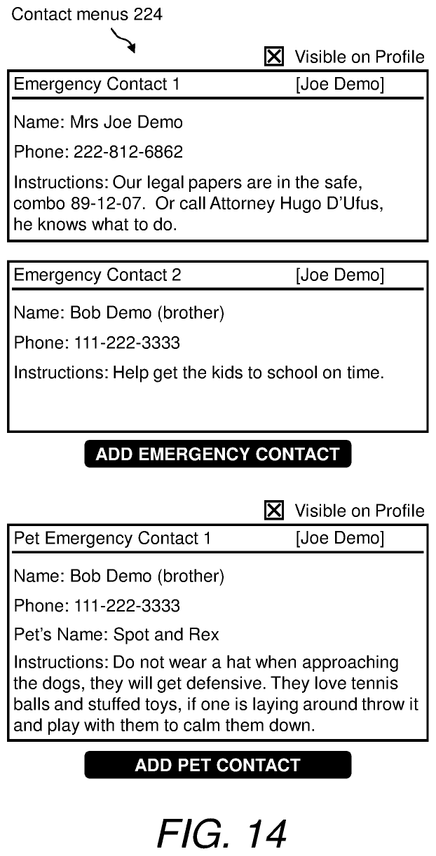

In the view shown in FIG. 13, the contacts tab in main menu 214 is selected and the viewing panel shows example pushbuttons: ALL CONTACTS, EMERGENCY CONTACTS, CHILD CONTACTS, and PET CONTACTS. Referring now to FIG. 14, a set of contacts menus 224 are shown. By way of example, emergency contact 1 and 2 panels that correlate to the EMERGENCY CONTACTS pushbutton and a pet emergency contact panel that correlates to the PET CONTACTS pushbutton. Again, each panel has a “Visible on Profile” check box.

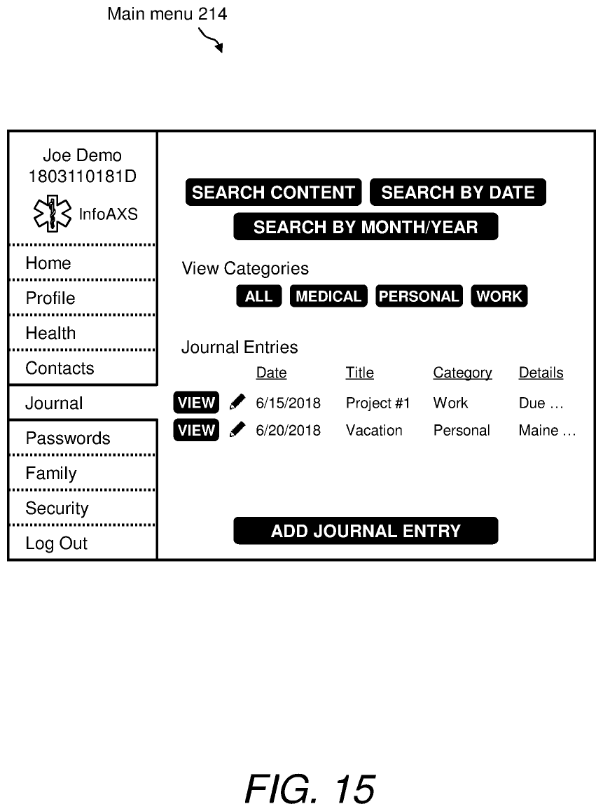

In the view shown in FIG. 15, the journal tab in main menu 214 is selected and the viewing panel shows an ADD JOURNAL ENTRY pushbutton. Using the ADD JOURNAL ENTRY pushbutton, a screen (not shown) is presented by which a user may enter, for example, the date, title, category, and content of the journal entry. Once saved, each journal entry may be displayed using the journal tab. Other control pushbuttons may be provided for navigating journal entries. For example, search pushbuttons: SEARCH CONTENT, SEARCH BY DATE, and SEARCH BY MONTH/YEAR. Further, other control pushbuttons may be provided for viewing journal entries by category. For example, search pushbuttons: ALL, MEDICAL, PERSONAL, and WORK. A user can select a given journal entry by pressing the VIEW pushbutton next to the desired entry.

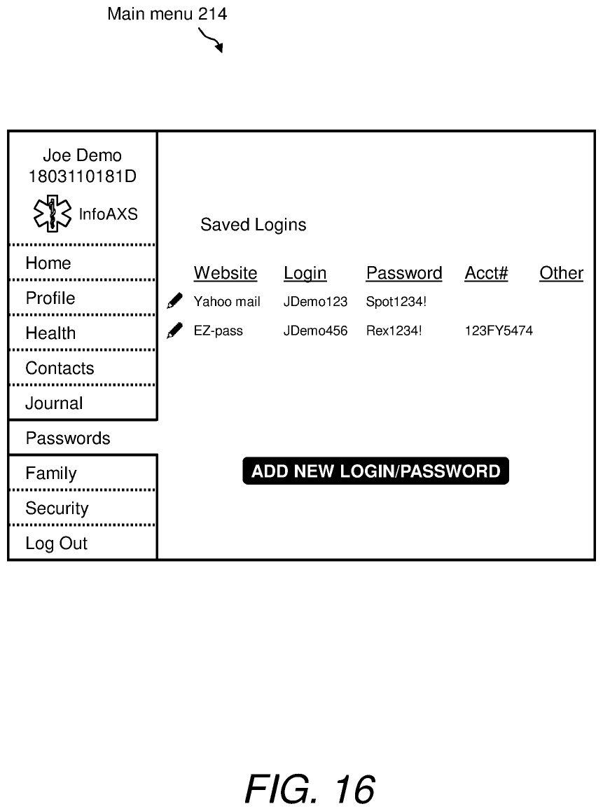

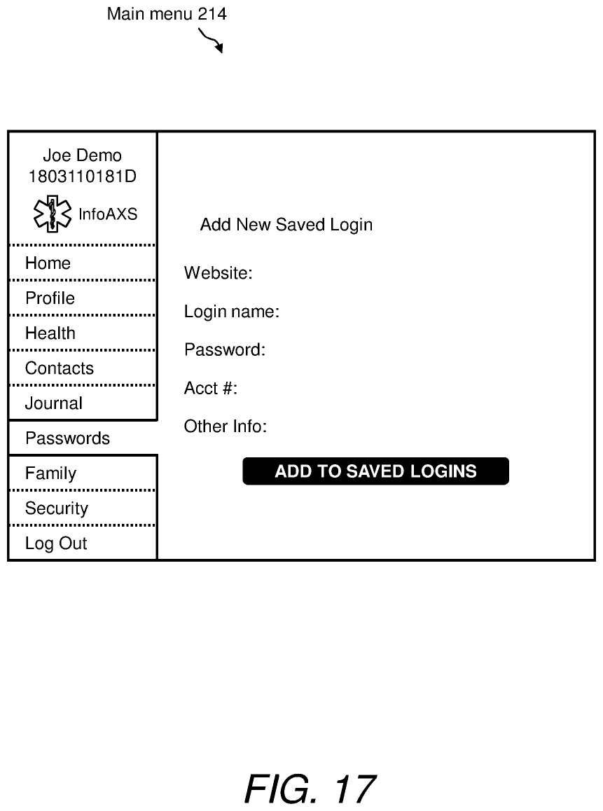

In the view shown in FIG. 16, the passwords tab in main menu 214 is selected and the viewing panel shows, for example, an ADD NEW LOGIN/PASSWORD pushbutton. Using the ADD NEW LOGIN/PASSWORD pushbutton, the screen shown in FIG. 17 is displayed wherein the user may enter various login/password information. Once the information is entered, the information may be saved by pressing an ADD TO SAVED LOGINS pushbutton. Referring now again to FIG. 16, a list of saved login/password information is presented.

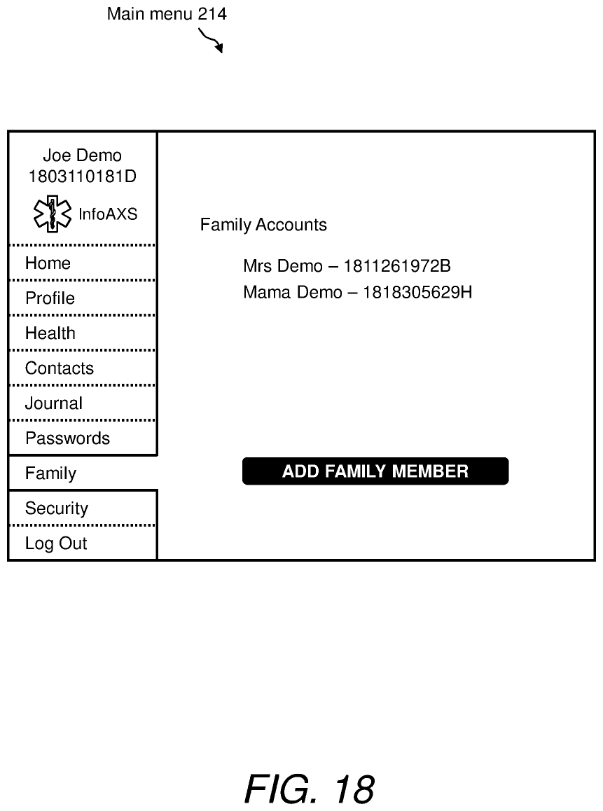

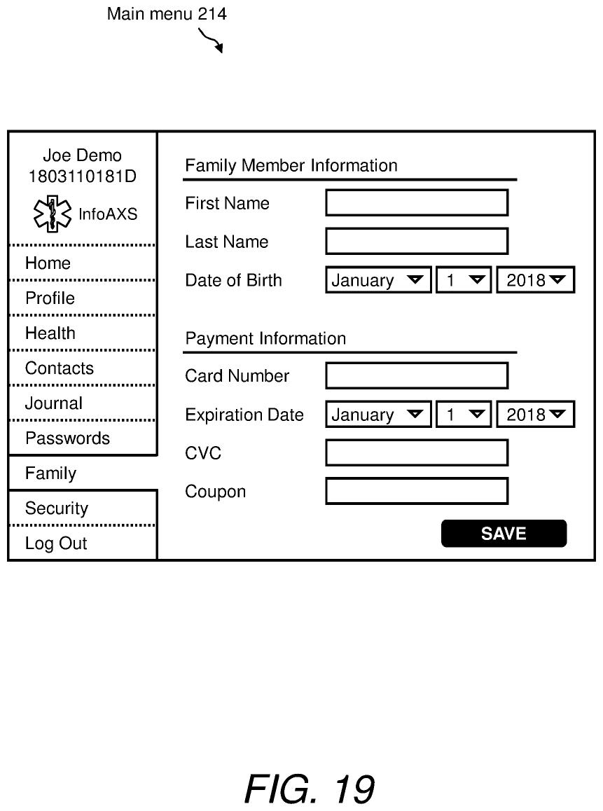

In the view shown in FIG. 18, the family tab in main menu 214 is selected and the viewing panel shows, for example, an ADD FAMILY MEMBER pushbutton. Using the ADD FAMILY MEMBER pushbutton, the screen shown in FIG. 19 is displayed wherein the user may enter the name and birthday of a family member and, if needed, payment information for that family member, and/other information. Once the information is entered, the information is saved by pressing an SAVE pushbutton. Referring now again to FIG. 18, a list of saved family members is presented.

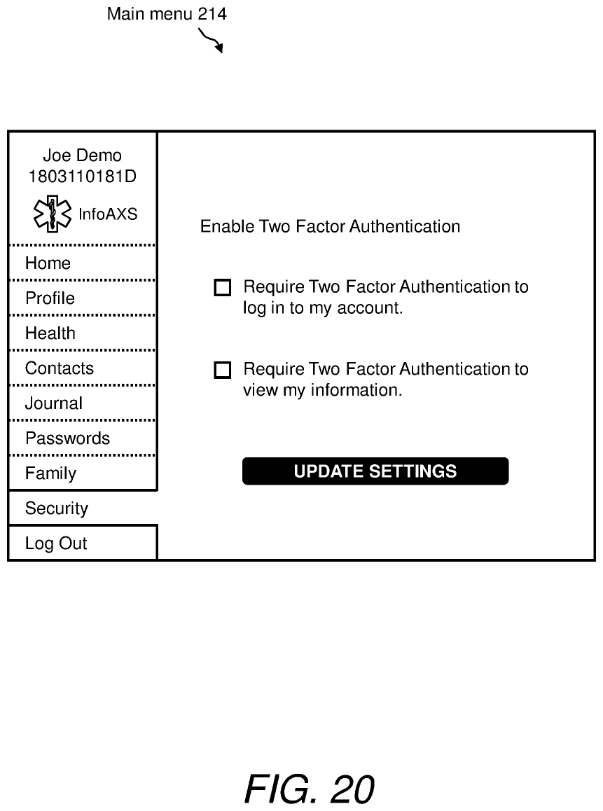

In the view shown in FIG. 20, the security tab in main menu 214 is selected and the viewing panel shows, for example, an UPDATE SETTINGS pushbutton. Here, the user my select, for example, whether he/she wishes to enable two factor authentication.

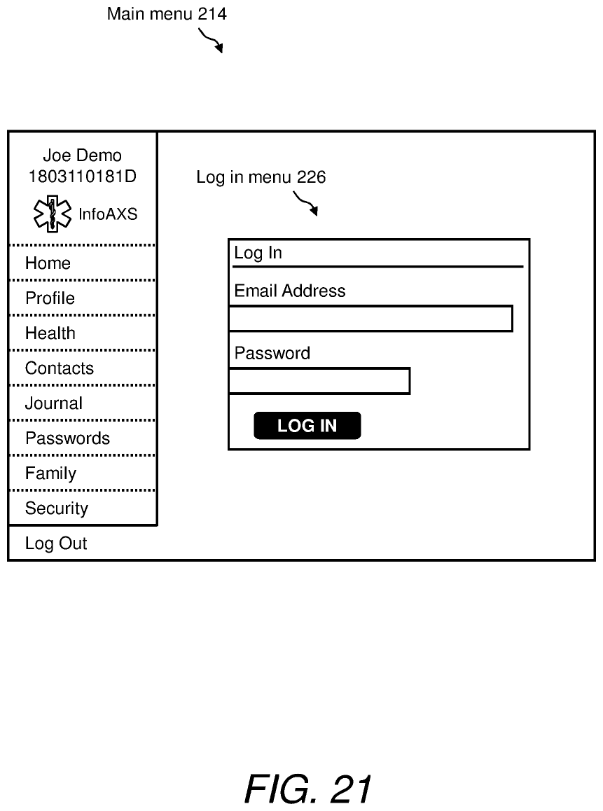

In the view shown in FIG. 21, the log out tab in main menu 214 is selected. By selecting the log out tab, the user is automatically logged out of information access system 100 and the viewing panel shows a log in menu 226 by which a user may log back into information access system 100.

Info access GUI 118/118′ of info access application 110 of information access system 100 is not limited to the tabs, menus, panels, and/or screens shown in FIG. 6 through 21. These tabs, menus, panels, and/or screens are exemplary only and not meant to be inclusive of all the functions supported in information access system 100. Further, the tabs, menus, panels, and/or screens of info access GUI 118/118′ of info access application 110 of information access system 100 can be tailored for any applications and are not limited to heath and/or first responder applications. For example, the tabs, menus, panels, and/or screens of info access GUI 118/118′ of info access application 110 of information access system 100 can be tailored for any other applications, such as but not limited to, medical offices, schools, daycare centers, assisted living facilities, any other types of businesses as a means of maintaining clients' records, and the like

Further, in the tabs, menus, panels, and/or screens of info access GUI 118/118′ shown in FIG. 6 through 21, the information that is entered in any view that does not include the “Visible on Profile” check box is assumed to be “private” information. This may be for the convenience of the user. However, in other embodiments, each and every tab, menu, panel, and/or screen may include the “Visible on Profile” check box. In this case, the user must make a selection in each and every tab, menu, panel, and/or screen of info access GUI 118/118′ as to whether the information is to be classified public or private. Preferably, in the embodiment where, each and every tab, menu, panel, and/or screen includes the “Visible on Profile” check box, the default setting is preferably set to be private, e.g., the “Visible on Profile” check box is not checked.

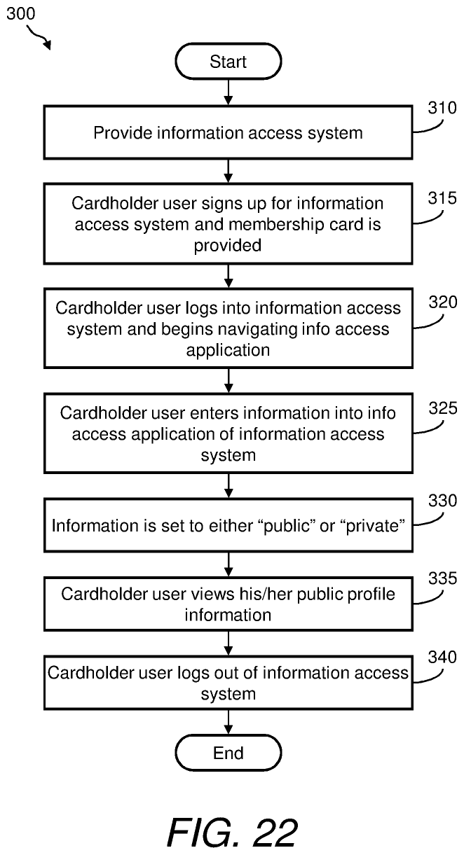

Referring now to FIG. 22 is a flow diagram of a method 300, wherein method 300 is an example of a method by which cardholder users 105 use the information access system 100. Method 300 may include, but is not limited to, the following steps.

At a step 310, information access system 100 is provided.

At a step 315, a cardholder user 105 signs up for or otherwise joins information access system 100 and a membership card 114 is provided to the cardholder user 105.

At a step 320, the cardholder user 105 logs into information access system 100 and begins navigating info access application 110 via info access GUI 118/118′.

At a step 325, cardholder user 105 enters information into info access application 110 of information access system 100. For example, using his/her cardholder computing device 150 and info access tool 110′, cardholder user 105 selects a certain tab, menu, panel, and/or screen of info access GUI 118/118′ and enters any desired information.

At a step 330, for each tab, menu, panel, and/or screen, the information is set to either “public” or “private.” For example, based on the selected tab, menu, panel, and/or screen and/or based on the selected state of any “Visible on Profile” check box, the information that is stored in cardholder data 132 is set to public information or is private information.

At a step 335, cardholder user 105 views his/her public profile. For example, if cardholder user 105 wishes to review the public information that is viewable by, for example, third-party users 107, cardholder user 105 may select the VIEW MY PAGE pushbutton shown in the profile tab of main menu 214 as shown in FIG. 7. In so doing, a viewable cardholder profile (e.g., viewable cardholder profile 212 shown in FIG. 5A and FIG. 5B) may be displayed for review with an option to print by clicking on a button.

At a step 340, upon completion of any desired operations within info access application 110, cardholder user 105 logs out of information access system 100.

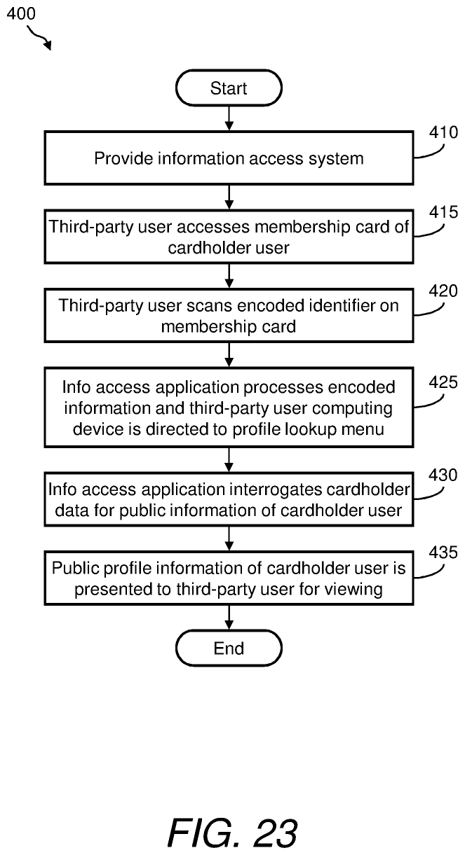

Referring now to FIG. 23 is a flow diagram of a method 400, wherein method 400 is an example of a method by which third-party users 107 use the presently disclosed information access system 100. Method 400 may include, but is not limited to, the following steps.

At a step 410, information access system 100 is provided.

At a step 415, a third-party user 107, such as a first responder, accesses membership card 114 of a certain cardholder user 105.

At a step 420, using his/her scanning device 154, third-party user 107, such as the first responder, scans encoded identifier 116 on membership card 114 of the cardholder user 105 of interest.

At a step 425, info access application 110 processes information of encoded identifier 116 and third-party computing device 152 may be directed to a profile lookup menu. For example, upon scanning encoded identifier 116, third-party computing device 152 may be automatically directed to the web address (e.g., www.infoaxs.com) of info access application 110 of information access system 100 and to, for example, third-party access menu 210 shown in FIG. 4. In one example, the unique ID number of the cardholder user 105 autofills (or auto-fills) the “Enter Member ID” field of third-party access menu 210. In another example, third-party user 107 manually enters the ID number into the “Enter Member ID” field. Then, the “Look up Profile” pushbutton is selected.

At a step 430, info access application 110 interrogates the cardholder data 132 of the cardholder user 105 of interest for any and all information that is marked “public” information.

At a step 435, any and all public information of the cardholder user 105 of interest is compiled, processed, and presented to third-party user 107 for viewing or printing. In one example, viewable cardholder profile 212 shown in FIG. 5A and FIG. 5B is displayed to third-party user 107.

In summary and referring now again to FIG. 1 through FIG. 23, information access system 100 and info access application 110 and methods 300, 400 are provided, wherein cardholder users 105 may enter any type of information and wherein some portion of the information can be public information and another portion of the information can be private information. Generally, in info access application 110 and methods 300, 400, all information is classified private by default unless specifically marked public by the cardholder user 105. Further, each cardholder user 105 has a membership card 114 with an encoded identifier 116, such as a QR code or barcode, printed thereon or RFID tag, and wherein a third-party user 107 can scan encoded identifier 116 and view the public information of the cardholder user 105.

Further, whereas information access system 100 and info access application 110 and methods 300, 400 may be tailored for use in, for example, a health application and more particularly for use in a first responder application, any other types of information (albeit unrelated to health) may be stored, processed, and/or conveyed using information access system 100.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A system comprising:
a. a hardware processor; and
b. a memory device, the memory device storing instructions, the instructions when executed causing the hardware processor to perform operations, the operations comprising:
i. receiving a member unique identification code, wherein the member unique identification code is accessed from an encoded identifier device;
ii. querying an electronic database for member data associated with the member unique identification code;
iii. retrieving at least a portion of the member data associated with the member unique identification code; and
iv. displaying the retrieved member data.

2. The system of claim 1, further comprising an encoded identifier on or in the encoded identifier device, wherein the encoded identifier is associated with the member unique identification code.

3. The system of claim 2, further comprising a third-party user device configured to read the encoded identifier to access the member unique identification code.

4. The system of claim 2, wherein the encoded identifier comprises at least one of a quick response (QR) code, barcode, RFID tag, or alphanumeric code.

5. The system of claim 3, wherein the third-party user device is configured to receive and display the retrieved member data.

6. The system of claim 2, wherein the encoded identifier device comprises a member user card, wherein the encoded identifier comprising at least one of a quick response (QR) code, barcode, RFID tag, or alphanumeric code.

7. The system of claim 1, further comprising a member user device, wherein the member user device is configured to one or more of input, edit, and/or access the member data.

8. The system of claim 5, wherein the operations further comprise sending the retrieved member data to the third-party user device.

9. The system of claim 5, wherein the operations further comprise designating certain portions of the member data as public, and wherein only the member data designated as public is displayable on the third party device.

10. A system, comprising:
a. an encoded identifier device;
b. a third-party device configured to read an encoded identifier on or in the encoded identifier device;
c. a hardware processor; and
d. a memory device, the memory device storing instructions, the instructions when executed causing the hardware processor to perform operations, the operations comprising:
i. receiving a member unique identification code from the third-party device, wherein the member unique identification code is accessed by the third-party device by reading the encoded identifier of the encoded identifier device;
ii. querying an electronic database for member data associated with the member unique identification code;
iii. retrieving at least a portion of the member data associated with the member identification code; and
e. displaying the retrieved member data to the third-party device.

11. The system of claim 10, wherein the encoded identifier device comprises an encoded identifier comprising at least one of a quick response (QR) code, barcode, RFID tag, or alphanumeric code.

12. The system of claim 10, wherein the encoded identifier device comprises a member user card, wherein the encoded identifier comprising at least one of a quick response (QR) code, barcode, RFID tag, or alphanumeric code.

13. The system of claim 10, further comprising a member user device, wherein the member user device is configured to one or more of input, edit, and/or access the member data.

14. The system of claim 10, wherein the operations further comprise designating certain portions of the member data as public, and wherein only the member data designated as public is displayed to the third-party device.

15. A method, comprising:
a. receiving, by a server, a service request sent via the Internet from a client device, the service request requesting a cloud-based data retrieval service performed on behalf of the client device, the service request specifying a member unique identification code;
b. querying, by the server, an electronic database for member data associated with the member unique identification code, the electronic database electronically associating the member unique identification code with the associated member data;
c. retrieving, by the server, at least a portion of the associated member data;
d. sending, by the server, the retrieved member data via the Internet to the client device; and
e. displaying the retrieved member data.

16. The method of claim 15, wherein the member unique identification code is accessed from an encoded identifier disposed on or in an encoded identifier device.

17. The method of claim 16, wherein the client device comprises a third-party device configured to read the encoded identifier of the encoded identifier device.

18. The method of claim 15, wherein the retrieved member data comprises only member data designated as public.

19. The method of claim 16, wherein the encoded identifier comprising at least one of a quick response (QR) code, barcode, RFID tag, or alphanumeric code.

20. The method of claim 15, wherein the client device comprises a member user device, wherein the member user device is configured to one or more of input, edit, and/or access the member data.
